(19) United States
(12) Patent Application Publication
(10) Pub. No.: US 2016/0361250 A1
(43) Pub. Date: Dec. 15, 2016

(54) ACTIVE INGREDIENTS STIMULATING TYPE 2 AND/OR TYPE 3 HUMAN BETA-DEFENSINS AND COSMETIC OR PHARMACEUTICAL COMPOSITIONS CONTAINING SUCH ACTIVE INGREDIENTS

(71) Applicants: BASF Beauty Care Solutions France S.A.S., Lyon (FR); Yves Saint Laurent Parfums, Neuilly-sur-Seine (FR)

(72) Inventors: Eric Perrier, Les Cotes D'Arey (FR); Anne Guezennec, Croissy Sur Seine (FR); Ingrid Pernet, Grezieu La Varenne (FR); Corinne Reymermier, Charly (FR); Joelle Guesnet, Bures Sur Yvette (FR)

(21) Appl. No.: 15/248,195

(22) Filed: Aug. 26, 2016

Related U.S. Application Data
(63) Continuation of application No. 10/308,259, filed on Nov. 25, 2002.

(30) Foreign Application Priority Data
Aug. 2, 2002 (FR) 02 09905

(51) Int. Cl. A61K 8/97; A61K 36/185; A61K 36/36; A61K 36/88; A61K 36/889; A61K 36/49; A61K 36/42; A61K 36/21; A61K 31/19; A61K 31/223; A61K 8/44; A61K 8/36; A61Q 19/08; A61Q 19/02; A61Q 1/12; A61Q 17/04; A61Q 19/00; A61Q 5/02; A61K 36/28

(52) U.S. Cl. CPC A61K 8/97; A61K 36/28; A61K 36/185; A61K 36/36; A61K 36/88; A61K 36/889; A61K 36/49; A61K 36/42; A61K 36/21; A61K 31/19; A61K 31/223; A61K 8/44; A61K 8/36; A61Q 19/08; A61Q 19/02; A61Q 1/12; A61Q 17/04; A61Q 19/007; A61Q 5/02

(57) ABSTRACT

The invention relates to active ingredients capable of stimulating direct or indirect expression of type 2 human beta-defensins and/or type 3 human beta-defensins. The invention thus provides an active ingredient capable of stimulating direct or indirect expression of type 2 human beta-defensins and/or type 3 human beta-defensins, characterized in that the active ingredient does not cause any inflammatory, irritation or intolerance reactions. The invention also provides a screening method for the selection of such active ingredients. The invention is applicable to the preparation of cosmetic or pharmaceutical compositions containing such active ingredients.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_074050_0052_01. The size of the text file is 2 KB, and the text file was created on Aug. 26, 2016.

This invention relates principally to active ingredients able to stimulate direct or indirect expression of type 2 and/or type 3 human beta-defensins. This invention relates principally to cosmetic or pharmaceutical compositions containing these active ingredients. This invention relates principally to a treatment method using such compositions. This invention relates principally to a screening method to select such active ingredients.

### STATE OF THE ART

Antibiotic peptides are small molecules (10 to 50 amino acids) able to destroy microorganisms such as bacteria, fungi or viruses by permeabilising their cell membrane. Since their discovery in arthropods in 1981, nearly 500 molecules with such properties have been identified in higher organisms (plants, insects, mammals, humans). The property common to antimicrobial peptides is their amphiphathic structure, that is the distribution of amino acids into cationic (positively charged) zones distinct from hydrophobic zones. It is this structure which confers on them their activity and specificity of action against bacterial cytoplasm membranes, since the membranes have a large number of negatively charged anionic phospholipids which bind to the cationic sites of antibiotic peptides (Zalsoff M. Antimicrobial peptides of multicellular organisms. Nature 2002; 415 : 389-395). Depending on their structure, the scope of activity of these peptides can be either very narrow or very broad.

Antimicrobial peptides are found mainly in the animal world: in venoms (scorpions, bees, spiders), in drosophila or bluefly, in bovine neutrophils, in leukocytes and porcine small intestine, in milk, in frogs, Mediterranean mussels, etc. In the plant world, thionins and defensins protect the plants seeds and vegetative tissues against bacteria.

Most antibacterial peptides are found in the epithelial tissues of animals where they play an important part as first immune barrier. They have been found in the skin of frogs and mice, as well as in the gastrointestinal and respiratory systems of humans and in human skin and mucosa.

The defensins are one of the most studied class of antimicrobial peptides. This class consists of cysteine-rich molecules with three disulphide bridges. They are found in plants, insects and various mammals. In humans, two classes of defensin are found which differ from one another in terms of spacing and bonds between the six cysteine residues:
the α-defensins (6 types) isolated from neutrophils (HNP1 to 4, Human Neutrophil Peptide) and in the Paneth cells of the gastrointestinal tract (α-defensins 5 and 6)
the β-defensins, longer and more basic, expressed in the mucosa and epithelial cells (skin, trachea, tongue, tonsils, saliva . . . ) occur in 3 forms:
a constitutive form, hBD1 (Human β-defensin 1), found mainly in the kidneys but also in the pancreas, saliva, lungs, placenta and skin.
Two inducible forms: hBD2 and hBD3 (Human β-defensin 2 and 3):
hBD2 is expressed in the skin, trachea, and lungs; its expression is triggered by bacterial stimulation, TNFα (Tumour Necrosis Factor) (Harder J. et al., A peptide antibiotic from human skin. Nature 1997; 387 : 861), LPS (Lipopolysaccharides) (Matthews E. et al., Production of beta-defensin antimicrobial peptides by the oral mucosa and salivary glands. Infect Immun 1999; 67 : 2740-2745), IL1α and IL1β (Interleukin 1) (Liu A Y et al., Human β-defensin-2 production in keratinocytes is regulated by interleukin-1, bacteria and the state of differentiation. J Invest Dermatol 2002; 118; 275-281), all these molecules having in common the property of being involved in inflammatory processes. The antibacterial activity of hBD2 is aimed in particular at Gram negative bacteria such as Escherichia coli.
hBD3 is found in the skin, trachea, tonsils and tongue (Harder J. et al., Isolation and characterization of human β-defensin-3, a novel human inducible peptide antibiotic. J Biol Chem 2001, 276 : 5707-5713), as well as in muscle tissue and the heart (Conejo Garcia et al., Identification of a novel, multifunctional β-defensin (human β-defensin-3) with specific antimicrobial activity. Cell tissue research 2001; 306 : 257-264). hBD3 is induced by bacterial stimulation, TNFα and especially IFNγ (gamma interferon) which also have the common property of being molecules involved in inflammatory processes. The spectrum of activity of hBD3 is broader than that of hBD2 as it is capable of lysing Gram negative and Gram positive bacteria such as Staphylococcus aureus.

Induction of these defensins in the skin is the body's first line of defence to protect itself against the increasing number of pathogenic microorganisms it is faced with. In addition, hBD2 has chemotactic properties for immature dendritic cells and memory T-lymphocytes (Yang D. et al., Betadefensins: linking innate and adaptative immunology through dendritic and T cell CCR6. Science 1999; 286 : 525-528) and thus seems to play an important part in promoting immune response, inflammation and healing.

The increase in the amount of β-defensins in human skin therefore contributes to preserving the cutaneous microbial ecosystem by preventing overinvasion by particular species which might be pathogenic (Staphylococcus aureus), keeping the skin protected and healthy. To do this, two strategies can be envisaged:
either topical application of synthetic peptides with sequences or structures similar to those of known antibiotic peptides described by the man skilled in the art.
or provoked induction of defensins by topical or oral application of an active ingredient capable of stimulating synthesis of these peptides in natural secretory cells.
   With the exception of the previously cited cytokines (TNFα, IFNγ, IL1), few products have been described as having the capacity to induce β-defensins, except in animals where L-Isoleucine induces expression of hBD3 in bovine renal epithelial cell lines (Fehlbaum P. et al., An essential amino acid induces epithelial β-defensin expression. PNAS 2000; 97 : 12723-12728; Patent WO0168085 Anderson M. et al., Method for stimulation of defensin production. Sep. 20, 2001). In humans, it has been found that keratinocyte differentiation stimulates the production of hBD2 (Liu A Y et al., Human β-defensin-2 production in keratinocytes is regulated by interleukin-1, bacteria and the state of differentiation. J Invest Dermatol 2002; 118; 275-281).

Studies concerning the presence of hBD2 and hBD3 defensins in the skin were conducted on skin cuts, explants, and cultured cells or reconstructed skins. Increases in the amount of defensins can be visualized by a number of techniques but none of these techniques has yet been applied to the search by screening for an active ingredient able to stimulate quantitatively hBD2 and hBD3 in human cells. Techniques conventionally used by the man skilled in the art cannot, in fact, be applied to the problem in hand, in other words searching for active ingredients able to stimulate human defensins in a quantitatively proven manner (lack of commercial antibodies which makes it impossible to carry out quantitative assays for proteins using ELISA methods; in situ hybridisation type molecular biology methods, Northern transfer, RT-PCR, which are more qualitative than quantitative; models of human cells in very complex three-dimensional cultures which cannot be used in screening methods).

Such an assay will be developed within the scope of the present invention.

### AIMS OF THE INVENTION

The inventors' aim was to identify active ingredients able to induce the expression of type 2 and/or type 3 β-defensins without triggering inflammatory reaction and, for example, without overstimulating the secretion of molecules which are usually expressed in inflammatory reactions.

To date, all the molecules described as having the ability to stimulate defensins in humans (TNFα, IL1, LPS, bacteria, IFNγ . . . ) have proinflammatory properties. They stimulate inflammation cytokines such as IL1α, IL8 or MIP3α.

The aim of the invention therefore is to resolve the new technical problem of providing an active ingredient able to stimulate direct or indirect expression of type 2 and/or type 3 human beta-defensins, without triggering inflammatory, irritation or intolerance reactions.

The aim of the invention is to solve the new technical problem of providing an active ingredient able to stimulate direct or indirect expression of type 2 and/or type 3 human beta-defensins, without stimulating or without appreciably stimulating direct or indirect synthesis of proinflammatory molecules or molecules usually co-expressed in the course of inflammatory processes.

The aim of the invention is therefore to solve the new technical problem of providing a composition containing at least one active ingredient as defined above.

The aim of the invention is to solve the new technical problem of providing a tissue or cell model making to possible to screen the active ingredients as defined above.

The aim of the invention is to solve the new technical problem of providing a screening method for the active ingredients as defined above.

The aim of the invention is to solve the new technical problem of providing a composition containing the active ingredient as defined above for use in the field of cosmetics or pharmacy.

This use is essentially carried in order to exert a bactericidal and/or fungicidal activity, preferably by topical application to tissues, for example skin, mucosa, hair and nails or scalp.

### DESCRIPTION OF THE INVENTION

The inventors use the term “active” to refer to potential active ingredients to be screened and the term “defensins” to refer to type 2 and/or 3 β-defensins when the term is used to refer to defensins stimulated by the active ingredients of this invention.

According to a first aspect, this invention relates to an active ingredient able to stimulate direct or indirect expression of type 2 human beta-defensins and/or type 3 human beta-defensins which does not trigger a reaction selected from inflammatory, irritation or intolerance reactions.

The inventors use the term “which does not trigger inflammatory, irritation or intolerance reactions” to mean that the use of these active ingredients does not produce any disturbing adverse effects for the user of the active ingredient. The inventors use the term “disturbing” to mean reactions such as rashes, itching, irritation, increased skin sensitivity, etc.

Advantageously, said active ingredient able to stimulate direct or indirect expression of type 2 human beta-defensins (hBD2) and/or type 3 human beta-defensins (hBD3) does not stimulate or does not stimulate substantially direct or indirect synthesis of proinflammatory molecules or molecules usually co-expressed in the course of inflammatory processes.

The inventors use the term “not stimulate, or does not stimulate substantially direct or indirect synthesis of proinflammatory molecules or molecules usually co-expressed in the course of inflammatory processes” to refer to the fact that said active ingredient does not stimulate or does not promote proinflammatory molecules or molecules usually co-expressed in the course of inflammatory processes, or stimulates them or promotes their stimulation only to a degree that is less than that induced by TNF alpha (TNFα) or gamma interferon (IFNγ), preferably a stimulation below 75% of maximum stimulation induced by TNF alpha or gamma interferon in identical temperature, contact time and operating conditions.

Advantageously, said proinflammatory molecule or molecule usually co-expressed in the course of inflammatory processes is especially MIP 3 alpha or IL8 or IL1, or another interleukin or histamine, tryptase, P substance, leucotrienes or prostaglandins.

Advantageously, said active ingredient does not stimulate or does not stimulate substantially direct or indirect production of proinflammatory molecule(s) or molecule(s) usually co-expressed in the course of inflammatory processes in cultures of human epithelial cells, in particular keratinocytes and/or corneocytes in culture, or in human skin biopsies maintained in survival, or in models of reconstructed epidermis or in models of reconstructed skins, constructed or not on cell matrices or on de-epidermised dermises. Said epithelial cells are preferably “normal”.

The inventors use the term “normal” to mean epithelial cells not originating from non-immortalised cell lines but which can be cells originating from pathological tissues or genetically modified cells.

Advantageously, said active ingredient is chosen from artemisia root, Canadian erigeron, elderberry bark, rupturewort, pineapple juice, peppermint, areca, cocoa, quinoa, arnica, boldo, sarsaparilla, walnut leaf, hibiscus flower, pumpkin, sunflower, peony, St John's Wort, horse chestnut or one of their extracts, jasmonic acid or vitamin A, derivatives and precursors thereof, alpha-MSH or one of the peptides making up alpha-MSH or a chemical structure mimicking one of the peptides; isoleucine esters; calcium or any organic or mineral salt of calcium.

Advantageously, said active ingredients originating from plants are obtained by extraction, preferably by aqueous extraction but also by other methods of extraction such as in a water/butylene glycol mixture for example (from 1/99 to 99/1 by w/w). Jasmonic acid or A vitamin, derivatives and precursors thereof, alpha-MSH or one of the peptides making up alpha-MSH or a chemical structure mimicking one of these peptides, isoleucine esters and calcium or any organic or mineral calcium salts are used as such, diluted in water or ethanol or other solvents compatible with the model described.

According to a second aspect, the invention relates to a composition containing at least one active ingredient as defined above.

Advantageously, said composition contains said active ingredient at a concentration in the range of 0.001% to 20%, preferably 0.01% to 10%. These concentrations are expressed as a percentage by weight, in particular when this percentage is not clearly mentioned as being a percentage by weight.

The concentration is optimised such that the active ingredient is able to stimulate the expression of type 2 and/or type 3 human beta-defensins, while not inducing any irritation or inflammation, in particular not stimulating, or not stimulating substantially direct or indirect synthesis of proinflammatory molecules or molecules usually co-expressed in the course of inflammatory processes.

According to a third aspect, the invention relates to the use of at least one active ingredient as defined above for the manufacture of a cosmetic composition, said active ingredient being present in a concentration able to stimulate the expression of type 2 and/or type 3 beta-defensins and which does not induce any irritation or inflammation, and in particular which does not stimulate or not stimulate substantially direct or indirect synthesis of at least one proinflammatory molecule or molecule usually co-expressed in the course of inflammatory processes, said active ingredient possibly being mixed with a cosmetically acceptable excipient.

According to a fourth aspect, the invention relates to the use of at least one active ingredient as defined above for the manufacture of a pharmaceutical composition, said active ingredient being present in a concentration able to stimulate the expression of type 2 and/or type 3 beta-defensins and which does not induce any irritation or inflammation, and in particular which does not stimulate or not stimulate substantially direct or indirect synthesis of at least one proinflammatory molecule or molecule usually co-expressed in the course of inflammatory processes, said active ingredient possibly being mixed with a pharmaceutically acceptable excipient.

Advantageously, said composition includes at least one microbiocidal and/or microbiostatic agent. The inventors use the term “microbiocidal agent” for referring to the group of agents with a destructive effect on bacteria and the term “microbio static agent” for referring to the group of agents with a limitation effect on bacterial proliferation.

According to a fifth aspect, the invention relates to a cell or tissue model allowing screening of at least one active ingredient able to stimulate the expression of type 2 and/or type 3 beta-defensins and which does not induce irritation or inflammation, and in particular which does not stimulate or not stimulate substantially direct or indirect synthesis of at least one proinflammatory molecule or molecule usually co-expressed in the course of inflammatory processes.

Advantageously, said cell or tissue model includes epithelial cells, for example keratinocytes and/or corneocytes suitable for culturing under appropriate culture conditions with, in particular, a calcium content in the range of 0 to 100 mM, preferably 1.7 mM.

According to a sixth aspect, the invention relates to a screening method for active ingredients able to stimulate direct or indirect expression of type 2 and/or type 3 human beta-defensins and where said active ingredients do not induce irritation or inflammation, and in particular which do not stimulate or not stimulate substantially direct or indirect synthesis of at least one proinflammatory molecule or molecule usually co-expressed in the course of inflammatory processes (these active ingredients are described in this invention as “potential active ingredients to be screened”), comprised of the following steps:

a) culturing a cell or tissue model in the presence of various potential active ingredients to be screened under suitable culture conditions; the method can be applied to at least one potential active ingredient to be screened but it is more advantageous to test a large number of active ingredients (“or actives”) at the same time;

b) direct or indirect identification of the presence or not of stimulation of type 2 and/or type 3 human beta-defensin expression:

c) identification of the non-irritant and non-inflammatory properties of these active ingredients, in particular by lack of stimulation of direct or indirect synthesis of proinflammatory molecules or molecules usually co-expressed in the course of inflammatory processes.

Advantageously, said screening method comprises an additional step d) where at least one active ingredient, able to stimulate direct or indirect expression of type 2 and/or type 3 human beta-defensins, and simultaneously which does not stimulate or not stimulate substantially direct or indirect synthesis of proinflammatory molecules or molecules usually co-expressed in the course of inflammatory processes, is selected.

Readers will evidently be aware that the inventors do not limit their invention to particular methods of analysis which are simply a means to carry out the screening principle of this invention.

In fact, in future, other methods of analysis may replace the methods used by the inventors in this invention, such as methods using antibodies which recognize hBD2 and 3 (ELISA type methods, immunoblotting, immunohistology and so on).

Advantageously, said screening method includes in step a) a cell or tissue model containing epithelial cells or human skin biopsies maintained in survival, or models of reconstructed epidermis or models of reconstructed skin containing epithelial cells, for example keratinocytes and/or corneocytes suitable for culturing under appropriate culture conditions with, in particular, a calcium content in the range of 0 to 100 mM, preferably 1.7 mM.

Advantageously, said screening method includes, in step a), the presence of various potential active ingredients to be screened with the cell or tissue model for a period of 6 to 48 h, preferably about 16 h (contacting time).

Advantageously, said screening method includes in step b) extraction of total RNA and RT-PCR analysis of the expression of mRNA coding for the above-cited type 2 and/or 3 human beta-defensins.

Advantageously, said screening method includes in step b) a RT-PCR on actin (weakly stimulated reporter gene) such that increases in mRNA coding for hBD2 and hBD3 are referred to mRNA coding for actin.

Advantageously, said screening method includes in step b) depositing amplified mRNA on agarose gel containing a nucleic acid insertion visualisable under UV (such as ethidium bromide).

Advantageously, said screening method includes in step b) after migration of the products on agarose gel a comparison of the intensity rartios of hBD2/actin and hBD3/actin bands under UV light.

Advantageously, said screening method includes in step c) an ELISA-type assay for proinflammatory molecules or molecules usually co-expressed in the course of inflammatory processes.

Advantageously, said screening method includes in step d) selection of at least one active ingredient able to stimulate the expression of mRNA coding for said type 2 and/or 3 human beta-defensins without stimulating or stimulating substantially direct or indirect synthesis of proinflammatory molecules or molecules usually co-expressed in the course of inflammatory processes.

Advantageously, said screening method includes the selection of active ingredients stimulating the expression of the above-mentioned human beta-defensins and which does not stimulate or not stimulate substantially at least one proinflammatory molecule or at least one molecule usually co-expressed in the course of inflammatory processes, such as IL1, IL8, and MIP3α.

Advantageously, said screening method is comprised of the following steps:
contacting the potential active ingredients to be screened with normal human keratinocytes in monolayer in a specific medium without serum and enriched with calcium, for example by setting up an untreated control and two positive controls in parallel (TNFα for hBD2 and IFNγ for hBD3);
extracting then assaying total RNA using a spectrophotometer, preferably at a wavelength in the range 260 to 280 nm.; these RNA may be diluted if need be;
RT-PCR on actin, hBD2 and hBD3 is carried out on initial total RNA;
retrotranscription of total RNA then amplification of the retrotranscribed product (cDNA), carried out for example in a thermocycler and is according to an amplification programme which may be common to actin, hBD2 and hBD3;
mixing the hBD2, hBD3 and actin amplification products followed by addition of a mixture of a charge buffer and water (⅔), the final solution being deposited on premoulded agarose gel containing ethidium bromide. The samples migrate and the bands obtained by means of this technique can be visualised under UV, preferably in a dark room, and digitally photographed. Bands are analysed at this stage in order to quantify their intensity. As the basal level of defensin expression (untreated control) is not detectable, the intensity ratios of the hBD2/actin and hBD3/actin bands can be compared, for example with those obtained for the positive control (treated with TNFα for hBD2 and IFNγ for hBD3) and show any stimulation of the expression of the β-defensin in question.

Advantageously this screening method includes an additional step:
selection of potential active ingredients to be screened having exerted an effect on type 2 and/or 3 human beta-defensin expression. The supernatants corresponding to the actives are tested in order to determine the levels of molecules usually co-expressed in the course of proinflammatory processes such as MIP3α, IL1 and IL8 secreted in the culture medium under the effect of these actives. For example, the levels can then be referred to the assayed RNA concentration in order to compare results with each other.

Advantageously, said screening method makes it possible to detect that an active ingredient, able to stimulate, direct or indirect, expression of type 2 and/or 3 human beta-defensins, simultaneously does not stimulate, or not stimulate substantially, direct or indirect synthesis of proinflammatory molecules or molecules usually co-expressed in the course of inflammatory processes, when it is chosen from among artemisia root, Canadian erigeron, elderberry bark, rupturewort, pineapple juice, peppermint, areca, cocoa, quinoa, arnica, boldo, sarsaparilla, walnut leaf, hibiscus flower, pumpkin, sunflower, peony, St John's Wort, horse chestnut or one of their extracts, jasmonic acid or vitamin A, derivatives and precursors thereof, alpha-MSH or one of the peptides making up alpha-MSH or a chemical structure mimicking one of the peptides; isoleucine esters; calcium or any organic or mineral salt of calcium.

According to a seventh aspect, the invention relates to use of an active ingredient for the manufacture of a cosmetic composition used to exert a bactericidal and/or fungicidal effect on treated tissues, in particular skin, mucosa, hair and nails or scalp. In the case of the scalp, said cosmetic composition can be used to prevent, to lower appearance of or treat dandruff.

According to an advantageous embodiment of the invention, the active ingredients subjects of the invention can be combined with other cosmetically active substances to reinforce protection of skin made fragile by the aging process and/or subject to stress caused by the environment and/or climate (cold, UV . . . ) and/or cleansing methods that are harsh on the cutaneous ecosystem. Compositions resulting from the invention can be formulated with any cosmetically acceptable excipient, in any galenic form used in cosmetics, in particular, in the form of an aqueous solution, possibly gelled, a lotion-type dispersion, possibly a two-phase dispersion, a water/oil or oil/water emulsion, a triple emulsion or a vesicle dispersion, creams, milks, liquid soaps, body emulsion, dermatological bars. The appearance of this composition can be dry or more or less fluid, or a white or coloured cream, a serum or a mousse. It may be applied to the skin in the form of an aerosol or stick. It can be used as a beauty and/or make-up product for the skin and/or as a cleansing product for the skin, mucosa, hair and nails and scalp. The composition resulting from the invention can also contain all the additives usable in cosmetics such as gelling agents, actives, preservatives, oils, solvents, antioxidants, scents, charges, pigments, filters, odour absorbers and dyes.

According to an advantageous embodiment of the invention, the active ingredients subjects of the invention can be combined with other pharmaceutically active substances to reinforce protection of skin made fragile by the aging process and/or subject to stress caused by the environment and/or climate (cold, UV . . . ) and/or cleansing methods that are harsh on the cutaneous ecosystem. Compositions resulting from the invention can be formulated with any pharmaceutically acceptable excipient, in any galenic form, in particular, in the form of an aqueous solution, possibly gelled, a lotion-type dispersion, possibly a two-phase dispersion, a water/oil or oil/water emulsion, a triple emulsion or a vesicle dispersion, creams, milks, liquid soaps, body emulsion, the appearance of this composition can be dry or more or less fluid, or a white or coloured cream, a serum or a mousse, it may be applied to the skin in the form of an aerosol or stick, and the composition resulting from the invention can also contain all the additives usable in pharmaceutics such as gelling agents, actives, preservatives, oils, solvents, antioxidants, scents, charges, pigments, filters, odour absorbers and dyes.

Advantageously, said composition incorporating at least one microbiocidal and/or microbiostatic agent is usable in particular in the cosmetics and pharmaceutical fields such that it combines the bactericidal effect of cutaneous beta-defensins with the effect of these agents, in particular to control cutaneous flora.

According to an eighth aspect, the invention relates to the use of an active ingredient for the manufacture of a pharmaceutical composition intended to exert bactericidal and/or fungicidal effect on treated tissues, in particular for the treatment and/or prevention or lowering appearance of acne and bacterial or fungicidal dermatosis.

Advantageously, said composition incorporating at least one microbiocidal and/or microbiostatic agent is usable, in particular, in the pharmaceutical field such that it combines the bactericidal effect of cutaneous beta-defensins with the effect of these agents, notably for the treatment of diseases related to a microbial agent such as dandruff, acne, vitiligo, dermatitis and other disorders of the skin, mucosa, scalp and nails caused by microbial agents.

The inventors have used analysis methods showing direct or indirect synthesis of proinflammatory molecule(s) or molecule(s) usually co-expressed in the course of inflammatory processes such that inflammatory, irritation and intolerance reactions can be identified.

The invention can be carried out in a different way to identify inflammatory, irritation or intolerance reaction(s), for example by evaluating itching, discomfort and tightness felt on application to the tissue to be treated.

According to another advantageous characteristic of the invention applicable for any one of these aspects, an active ingredient is selected which is able to stimulate direct or indirect expression of type 2 and/or type 3 human beta-defensin(s), and which does not stimulate, or not stimulate substantially, direct or indirect synthesis of proinflammatory molecules or molecules usually co-expressed in the course of inflammatory processes, for example a cytokine usually co-expressed during inflammatory process(es), such as IL1α, IL8 or MIP3α.

### DETAILED DESCRIPTION OF THE INVENTION

The method of the present invention developed by the inventors makes it possible to select by screening then identify active ingredients capable of increasing the quantity of hBD2 and/or hBD3 defensin mRNA in epidermal cells characterized in that said active ingredients do not cause, or do not cause substantially, inflammatory, irritation or intolerance reactions.

The screening method is comprised of the following steps:

Normal human epithelial cells, preferably normal human keratinocytes, are cultured as a monolayer in a specific medium devoid of serum, with a calcium concentration in the range of 0 to 100 mM, preferably 1.7 mM. At a given degree of confluence, preferably between 75% and 95%, preferably 80%, cells are contacted with the potential active ingredients to be screened for a period of time in the range of 6 to 48 h, preferably for 16 h. At least one untreated control and at least one positive control can be set up in parallel, preferably on the same culture plate, to facilitate screening. The positive controls are TNFα for hBD2 and IFNγ for hBD3 replacing the active ingredient to be screened. Advantageously, their concentrations are in the range of 1 to 500 ng/mL, preferably 100 ng/mL. After being contacted in this way, the supernatants are collected and the cells can be dry frozen, for example at −80° C., after rinsing in PBS (Phosphate Buffer Saline). Total RNA are extracted and assayed by spectrophotometry, preferably between 260 and 280 nm, and total RNA are preferably diluted to a concentration in the range of 2 to 50 ng/mL, preferably 5 ng/mL. Qualitative RT-PCR is performed on actin, hBD2 and hBD3. The primers used are taken from the literature (for hBD2: Harder J. et al., A peptide antibiotic from human skin. Nature 1997; 387 : 861 and for hBD2: Harder J. et al., Isolation and characterization of human β-defensin-3, a novel human inducible peptide antibiotic. J Biol Chem 2001, 276 : 5707-5713) and are:

-Actin:
sense:
(SEQ ID No. 1)
5′-GTGGGGCGCCCCAGGCACCA-3′
antisense:
(SEQ ID No. 2)
5′-CTCCTTAATGTCACGCACGATTTC-3′
-hBD2:
sense:
(SEQ ID No. 3)
5′-CCAGCCATCAGCCATGAGGGT-3′
antisense:
(SEQ ID No. 4)
5′-GGAGCCCTTTCTGAATCCGCA-3′
-hBD3:
sense:
(SEQ ID No. 5)
5′-AGCCTAGCAGCTATGAGGATC-3′
antisense:
(SEQ ID No. 6)
5′-CTTCGGCAGCATTTTCGGCCA-3′

The sequences of the primers used for RT-PCR can be different from those cited as long as they remain specific to the genes studied (actin, hBD2, hBD3).

RT-PCR is preferably carried out on a quantity of initial mRNA of 10 to 100 ng, preferably 50 ng, in a thermocycler, possibly according to a common programme. This step amplifies the initial RNA.

The temperature and time parameters for RT-PCR can change as a function of the primers or material used (thermocycler, RT-PCR kit supplier . . . ).

After amplification, the products are mixed together and a charge and water (⅔) buffer is added. The final solution is deposited on premoulded agarose gel containing a nucleic acid insertion visualisable under UV (such as ethidium bromide), at 2% for example. The samples migrate and the bands are visualised under UV in a dark room, and digitally photographed. Photos of the gel are analysed by image processing software which quantifies the band intensities. As the basal level of defensin expression (untreated control) is not detectable, the intensity ratios of the hBD2/actin and hBD3/actin bands can be compared, for example with those obtained for the positive control (treated with TNFα for hBD2 and IFNγ for hBD3) and make it possible to detect any stimulation of the expression of the β-defensin in question

At the end of this first step, the actives having exerted an effect on β-defensin expression are selected.

The supernatants corresponding to these actives are then tested using an ELISA kit in order to determine their content in MIP3α, IL1 and IL8 secreted into the culture medium under the effect of the actives. The concentrations assayed are referred to the RNA concentrations in order to compare results with each other. Potential active ingredients to be screened inducing an overstimulation of MIP3α, IL1 and IL8 (significantly higher than 75% of maximum stimulation by TNFα or IFNγ) are eliminated from the screening.

The gels are analysed by image processing software which quantifies the band intensities. Band visualisation can evidently be carried out on any nucleic acid electrophoresis system, as the type of insertion and the quantity of products resulting from RT-PCR can vary but remains below light saturation.

Validation of the results obtained can be carried out by the screening method of the invention applied to a “dose-effect” study with quantitative RT-PCR, which is described hereafter, but which is not limited to this particular method, because the man skilled in the art may regard other methods as suitable.

This real-time RT-PCR technique is the preferred method at present which gives quantifiable results concerning differences in mRNA expression.

A cytotoxicity study is carried out for the actives selected with increasing doses of 0.001% to 10%, preferably 0.01% to 10%. Viability must be set and is preferably in excess of 65%, and still preferably in excess of 75%. This viability sets the non-cytotoxic concentration limit.

The potential active ingredients to be screened were therefore tested on several concentrations, preferably from 0.001% to the limit non-cytotoxic concentration, on normal epithelial cells, preferably, normal human keratinocytes as a monolayer in a specific medium devoid of serum as described earlier.

The supernatants are then collected and the cells can be dry frozen at −80° C. after rinsing in PBS. Total RNA are extracted and diluted in the same concentration range as earlier. Dilute RNA are used for quantitative RT-PCR on actin, hBD2 and hBD3.

This technique is preferably performed on the same amounts of initial RNA as described earlier, preferably using a one-step kit containing SYBR® Green. However, the RT-PCR kit can be based on a technique other than SYBR® Green, such as Scorpion®, Molecular beacons®, Taqman™ probes, etc, advantageously in a fluorescence thermocycler with the same primers as above, whereby an amplification program is carried out, which can be identical to the previously described program.

Carrying out a study of fusion graphs makes it possible to verify the specificity of amplified products. The fluorescence graph as a function of the number of cycles gives the C(T) value corresponding to the number of cycles needed to obtain initiation of the fluorescence signal. The more an mRNA is expressed, the lower the C(T). Calculation of Sgene=(½)^{C(T)} for each RNA takes into consideration the exponential increase in the number of copies during amplification. The Sgene hBD2/Sgene actin and Sgene hBD3/Sgene actin ratios can be compared to an untreated control to give the percentage stimulation produced.

Untreated controls can be identical to those in the first step of the screening method according to the present invention.

The supernatants of the potential active ingredients to be screened, whose capacity to induce defensins has been confirmed, are tested maybe using an ELISA kit in order to assay MIP3α, IL8 and IL1α levels.

Preferably, these assays are performed on the supernatant. The levels are compared with the assayed RNA concentration of each sample. The non-inflammatory nature of the actives selected can thus be confirmed and/or the optimum dose for defensin stimulation without this inducing secretion of inflammation molecules can thus be found.

This invention also relates to active ingredients tested by this screening method since the inventor's principal objective was to discover active ingredients able to stimulate the expression of type 2 and/or type 3 β-defensins without stimulating said molecule secretion.

In a screening optic, a culture of normal human epithelial cells, preferably normal keratinocytes, is favoured when carried out on a 96-well plate. The expression of hBD2 and hBD3 defensins is very low in the case of undifferentiated basal keratinocytes and varies treatly as a function of donor and site of cell sampling. hBD2 in particular is found in 100% of facial skin or foreskin samples and only in 50% of abdominal or breast surgery samples (Ali R S et al., Expression of the peptides antibiotics hBD1 and hBD2 in normal human skin. J Invest Dermatol 2001; 117 : 106-111).

This invention makes it possible to provide a reproducible model, allowing a wide range of potential active ingredients to be tested and the expression of hBD2 and hBD3 mRNA to be detected.

This invention relates to a system for culturing keratinocytes in a calcium specific medium. The differentiation induced under these culturing conditions makes it possible to increase the basal expression level of mRNA of hBD2 and hBD3 defensins and this facilitate detection of their stimulation.

Culturing cells in 96 wells is a model that allows the desired effect to be screened and qualitative and quantitative analyses were adapted to the 96-well format.

Qualitative RT-PCR makes it possible to select a wide range of actives and verification of these by quantative RT-PCR is an essential step in the validation of results. The positive controls for stimulation (TNFα for hBD2 and IFNγ for hBD3) gives an induction value for defensins and inflammation molecules, acting as a reference and validating the quantitative RT-PCR technique.

The assay in the supernatants of the level of secretion of marker molecules for inflammation allows to select non-inflammatory actives.

The above desired activity was found, by means of this screening method, in the following active ingredients: artemisia root, Canadian erigeron, elderberry bark, rupturewort, pineapple juice, peppermint, areca, cocoa, quinoa, arnica, boldo, sarsaparilla, walnut leaf, hibiscus flower, pumpkin, sunflower, peony, St John's Wort, horse chestnut or one of their extracts, jasmonic acid or vitamin A, derivatives and precursors thereof, alpha-MSH or one of the peptides making up alpha-MSH or a chemical structure mimicking one of the peptides; isoleucine esters; calcium or any organic or mineral calcium salts.

In the examples, any characteristic which appears to be novel with respect to the state of the art is an integral part of this invention and protection is applied for in terms of both function and general aspects.

Moreover, in the description and claims, all percentages are given as weight percentages and the temperature is in degrees Celsius unless otherwise stated.

### EXAMPLE 1

### 1^{st} Step in the Screening Method:

A 1% concentration of the actives is tested on normal human keratinocytes, as a monolayer, on 96-well culture plates, in a specific medium enriched with calcium and free of serum (final concentration 1.7 mM).

At 80% confluence, cells are contacted with the actives (1 active per well) for 16 h. An untreated control and 2 positive controls (TNFα 100 ng/mL for hBD2 and IFNγ 100 ng/mL for hBD3) are set up in parallel on the same culture plate.

After 16 h, the supernatants are collected and cells are dry frozen at −80° C. after rinsing in PBS.

Total RNA are extracted using a 96-well extraction kit on silica columns and assayed using a 96-well spectrophotometer at 260 and 280 nm. RNA are diluted to 5 ng/mL.

Qualitative one-step RT-PCR is performed on 50 ng of initial RNA in 96 wells, on actin, hBD2 and hBD3. The primers are used at a concentration of 0.5 μM and arc taken from the literature:—hBD2: sense: 5′-CCAGCCATCAGCCATGAGGGT-3′; hBD2 antisense 5′-GGAGCCCTTTCTGAATCCGCA-3′ (Harder J. et al., A peptide antibiotic from human skin. Nature 1997; 387 : 861); hBD3 sense: 5′-AGCCTAGCAGCTATGAGGATC-3′; hBD3 antisense: 5′-CTTCGGCAGCATTTTCGGCCA-3′; actin sense: 5′-GTGGGGCGCCCCAGGCACCA-3′; actin antisense: 5′-CTCCTTAATGTCACGCACGTTTC-3′ (Harder J. et al., Isolation and characterization of human β-defensin-3, a novel human inducible peptide antibiotic. J Biol Chem 2001, 276 : 5707-5713).

Samples are placed in a thermocycler and follow a common amplification program: 50° C., 30 min; 94° C., 2 min, (94° C., 30 s; 60° C., 30 s; 68° C., 30 s), 32 cycles for the defensins and 30 cycles for actin; 72° C., 10 min; 14° C., infinite.

After amplification, the products are mixed at a rate of 3 μL of actin amplification products+6 μL of hBD2 amplification products+6 μL of hBD3 amplification products. 5 μL of a mixture of charge buffer and water (⅔) are added and the final 20 μL are deposited on 2% premoulded agarose gel. The samples migrate in 30 minutes and the bands are visualised under UV in a dark room then digitally photographed.

Photos of the gels are analysed by image processing software which quantifies the intensity of the bands. As the basal level of defensin expression (untreated control) is not detectable, the intensity ratios of the hBD2/actin and hBD3/actin bands can be compared, for example with those obtained for the positive control (treated with TNFα for hBD2 and IFNγ for hBD3) and indicate any stimulation of the expression of the β-defensin in question

At the end of this first step, the actives having exerted an effect on β-defensin expression are selected and the supernatants corresponding to these actives are then tested using an ELISA kit in order to determine the levels of MIP3α, IL1 and IL8 secreted into the culture medium under the effect of the actives. The levels are then referred to the RNA concentration assayed in each well in order to compare results with each other.

### Result Tables for the First Step:

Since the basal level of defensin expression is generally not detectable, stimulation of hBD2 and hBD3 is expressed as a percentage of the positive controls (TNFα for hBD2 and IFNγ for hBD3). IL8 and MIP3α levels are given as pg/mL/RNA concentration (in ng/μL).

TABLE I
Effects of active ingredients on hBD2 expression

| Actives (used at 1% w/w) | HBD2 | MIP3α | IL8 |
|---|---|---|---|
| Control | 0% | 6.7 | 30.9 |
| TNFα | 100% | 33 | 186 |
| Spirulin | 137% | 28.8 | 87 |
| Quinoa flour | 85% | — | — |
| Artemisia root | 76% | 7.2 | 30.2 |
| Elderberry bark | 65% | 7.5 | 17.9 |
| Sunflower | 58% | 10.9 | 68.6 |
| Canadian erigeron | 50% | 6.1 | 29.4 |
| Pineapple juice | 50% | 7.6 | 39 |
| Rupturewort | 44% | 7.8 | 55 |
| Cocoa | 39% | 4.9 | 22.5+ |
| Pumpkin | 35% | 5.2 | 33.1 |
| Peppermint | 26% | 1 | 4.8 |
| Sarsaparilla root | 26% | 4.7 | 15.4 |
| Areca | 1% | 0 | 25.7 |
| Floral Arnica | 0% | 3.9 | 27.8 |
| Peony flower | 0% | 0 | 2 |
| St John's Wort | 0% | 2.81 | 19.37 |
| Horse chestnut | 0% | 0.6 | 36.7 |
| Boldo | 0% | 1.8 | 20.1 |
| Walnut leaf | 0% | 1.4 | 19.4 |
| Hibiscus flower | 0% | 6.5 | 29.3 |
| Basil leaf | 0% | — | — |
| Black China tea | 0% | — | — |
| Raspberry | 0% | — | — |
| L-Isoleucine 10 μg/mL | 0% | — | — |
| D,L-Isoleucine 100 μg/mL | 0% | 5.6 | 30.6 |
| Isoleucine Methyl ester 100 μg/mL | 16% | 3.8 | 7.7 |
| Jasmonic Acid 100 μg/mL | 7% | 6 | 25.3 |

TABLE II
Effects of active ingredients on hBD3 expression

| Actives (used at 1% w/w) | HBD3 | MIP3α | IL8 |
|---|---|---|---|
| Control | 0% | 6.7 | 30.9 |
| TNFα | 100% | 13.8 | 97 |
| Floral Arnica | 460% | 3.9 | 27.8 |
| Peony flower | 192% | 0 | 2 |
| St John's Wort | 126% | 2.81 | 19.7 |
| Horse chestnut | 103% | 0.6 | 36.7 |
| Boldo | 86% | 1.8 | 20.1 |
| Rupturewort | 73% | 7.8 | 55 |
| Hibiscus flower | 50% | 6.5 | 29.3 |
| Areca | 45% | 0 | 25.7 |
| Peppermint | 40% | 1 | 4.8 |
| Walnut leaf | 27% | 1.4 | 19.4 |
| Cocoa | 15% | 4.9 | 22.5 |
| Spirulin | 6% | 28.8 | 87 |
| Basil Leaf | 0% | — | — |
| Black China tea | 0% | — | — |
| Raspberry | 0% | — | — |
| Artemisia root | 0% | 7.2 | 30.2 |
| Canadian erigeron | 0% | 6.1 | 29.4 |
| Elderberry bark | 0% | 7.5 | 17.9 |
| Pineapple juice | 0% | 7.6 | 39 |
| Quino flour | 0% | 8 | 39 |
| Sarsaparilla root | 0% | 4.7 | 15.4 |
| Sunflower | 0% | 10.9 | 68.6 |
| Pumpkin | 0% | 5.2 | 33.1 |
| L-Isoleucine 10 μg/mL | 0% | — | — |
| D,L-Isoleucine 100 μg/mL | 0% | 5.6 | 30.6 |
| Isoleucine Methyl ester 100 μg/mL | 6% | 3.8 | 7.7 |
| Jasmonic Acid 100 μg/mL | 0% | 6 | 25.3 |

Among these actives, those satisfying the criteria for our first step, that is those that stimulate hBD2 and/or hBD3 without triggering the expression of MIP3 and IL8 cytokines, are: artemisia root, Canadian erigeron, elderberry bark, rupturewort, pineapple juice, peppermint, areca, cocoa, quinoa, arnica, boldo, sarsaparilla, walnut leaf, hibiscus flower, pumpkin, sunflower, peony, St John's Wort, horse chestnut or one of their extracts, jasmonic acid and its derivatives and precursors, isoleucine esters.

Spirulin stimulates hBD2 strongly but triggers IL8 or MIP3 secretion and was therefore not selected. Other actives do not stimulate the defensins.

L-Isoleucine and a number of derivatives were tested: no significant stimulation of human defensins 2 and 3 was found by qualitative RT-PCR.

### EXAMPLE 2

### 2^{nd} Step of the Screening Method:

The actives which best satisfied the criteria for the first step undergo a dose-effect analysis.

A study of the cytotoxicity of the selected actives is carried out on increasing doses of 0.01% to 10%. Viability of 75% is set as the limit for non-cytotoxic concentration (max viability %).

The actives are then tested on 5 concentrations (from 0.001% to max viability) in quadruplicate on normal human keratinocytes as a monolayer on 96-well culture plates, in a specific medium enriched with calcium and free of serum (CaCl₂ 1.7 mM) (same conditions as in example 1).

After 16 h, the supernatants are collected and cells are dry frozen at −80° C. after rinsing in PBS.

Total RNA are extracted using a 96-well extraction kit on silica columns and assayed using a 96-well spectrophotometer at 260 and 280 nm. RNA are diluted to 5 ng/μL.

Quantitative RT-PCR in 96 wells on actin, hBD2 and hBD3 is initially carried out on 50 ng of RNA, using a one-step kit containing Sybrgreen, in a fluorescence thermocycler with the same primers as previously (0.5 μM). The amplification program is as follows: 50° C., 30 min; 94° C., 15 min; (94° C., 15 s; 60° C., 30 s; 72° C., 30 s)×50 cycles; 90° C., 1 min; 30° C., 1 min; 50° C. to 95° C. (10 s every ° C.); 14° C., infinite.

A study of the fusion graphs makes it possible to verify the specificity of amplified products. The fluorescence graph as a function of the number of cycles gives the C(T) value corresponding to the number of cycles needed to obtain initiation of the fluorescence signal. The more an mRNA is expressed, the lower the C(T) value. Calculation of Sgene=(½)^{C(T)} for each RNA takes into consideration the exponential increase in the number of copies during amplification. The Sgene hBD2/Sgene actin and Sgene hBD3/Sgene actin ratios can be compared with those of the untreated control to give the percentage stimulation produced.

The supernatants of the actives, whose capacity to induce defensins has been confirmed, are tested using an ELISA kit in order to assay MIP3α, IL8 and IL1α levels. These assays are performed on the same supernatant (200 μL) by carrying out a series dilution (by 1.5 to assay MIP3α and IL8, then by 2 to assay IL1). The levels are then referred to the RNA concentration assayed in each well in order to compare results with each other.

The inventors are thus able to confirm the non-inflammatory nature of the actives selected and/or find the optimum dose for defensin stimulation without this triggering the secretion of inflammation cytokines.

### Result Tables for the Second Step:

Quantitative RT-PCR makes it possible to obtain a basal value for the defensin expression for untreated controls. The results are therefore expressed as a percentage of the control. IL8, IL1 and MIP3 cytokine levels are given as pg/mL/RNA concentration (in ng/μL).

Dosis-effects of the actives selected in the first step (Table III)

| Active ingredient | Conc. | Viability | hBD2 | hBD3 | MIP3α | IL8 | IL1α |
|---|---|---|---|---|---|---|---|
| Control | | 100% | 100% | 100% | 5.6 | 47.4 | 26.9 |
| TNFα | 100 ng/mL | — | 1755% | 774% | 23.6 | 175 | 21.8 |
| IFNγ | 100 ng/mL | — | 472% | 4631% | 11.6 | 100.5 | 40.1 |
| Boldo | 0.1% | 84% | 182% | 168% | 2.2 | 33.6 | 28.2 |
| | 0.5% | 87% | 92% | 213% | 1.45 | 27.5 | 25.3 |
| | 1% | 76% | 72% | 703% | 0.4 | 31.7 | 20.2 |
| Arnica | 0.01% | 93% | 117% | 173% | 4.6 | 51.2 | 30.1 |
| | 0.1% | 91% | 128% | 561% | 2.5 | 34.1 | 38.5 |
| | 0.3% | 75% | 138% | 1830% | 4.1 | 24.1 | 37.9 |
| Quinoa | 0.1% | 83% | 143% | 102% | 6 | 58.7 | 23.4 |
| | 1% | 91% | 264% | 112% | 5.8 | 50.2 | 17.7 |
| | 5% | 90% | 401% | 237% | 12.4 | 150.2 | 34.4 |
| | 10% | 92% | 92% | 438% | 7.6 | 102.8 | 40.8 |
| Artemisia | 0.1% | 87% | 88% | 103% | 3.2 | 36.9 | 39.5 |
| | 1% | 75% | 117% | 116% | 2.9 | 35 | 34.9 |
| | 5% | 78% | 130% | 452% | 3.2 | 61.6 | 22.3 |
| | 10% | 83% | 104% | 3962% | 2.3 | 66.3 | 25.8 |
| Areca | 0.1% | 87% | 88% | 84% | 2.8 | 32.2 | 28.3 |
| | 1% | 77% | 35% | 261% | 0.28 | 23.7 | 39.5 |
| | 2% | 70% | 98% | 2781% | 0 | 7.6 | 44.8 |
| L-Isoleucine | 3.125 | — | 110% | 89% | 5.2 | 36.9 | 21 |
| μg/mL | 6.25 | — | 107% | 94% | 4.9 | 33.6 | 22.3 |
| | 12.5 | — | 95% | 101% | 5.9 | 42.2 | 25.3 |
| | 25 | — | 110% | 106% | 4.5 | 35.1 | 22.8 |
| Jasmonic Acid | 100 | — | 86% | 477% | 4.6 | 37.9 | 31.2 |
| μg/mL | 500 | — | 134% | 26919% | 13.6 | 103.3 | 97.1 |
| α-MSH ng/mL | 1 | — | 198% | 254% | 4.5 | 34.6 | 24.7 |
| | 100 | — | 186% | 217% | 3.9 | 41.7 | 26.1 |

This quantitative RT-PCR technique thus makes it possible to confirm the stimulatory effect of boldo, arnica and areca on hBD3 without inducing proinflammatory cytokines. 10% artemisia stimulates hBD3 without significantly stimulating the secretion of proinflammatory cytokines and Quinoa seed flour stimulates hBD2 from an active concentration of 1%. At 5%, it stimulates hBD2 and hBD3.

L-Isoleucine was tested on the 4 concentrations (3.125, 6.25, 12.5 and 25 μg/mL) described as being capable of stimulating bovine defensin-3 (Fehlbaum P. et al., An essential amino acid induces epithelial β-defensin expression. PNAS 2000; 97 : 12723-12728). This amino acid has not been found capable of inducing hBD2 and hBD3 expression in normal human keratinocytes.

100 μg/mL jasmonic acid and α-MSH are also capable of inducing defensins 2 and/or 3.

Among these actives, those satisfying the criteria of the invention, that is those that stimulate hBD2 and/or hBD3 without triggering the expression of MIP3, IL8 or IL1 cytokines, are: boldo, arnica, quinoa, artemisia or any of their extracts, jasmonic acid and its derivatives and precursors, αMSH or one of the peptides making up alpha-MSH or a chemical structure mimicking one of the peptides.

### EXAMPLE 3

In the same way as in example 2, retinoic acid and retinol were tested for their capacity to stimulate hBD2 and/or hBD3.

The results are as follows:

| Actives | Conc. | HBD2 | HBD3 |
|---|---|---|---|
| Control | | 100% | 100% |
| TNFα | 100 ng/mL | 1755% | 774% |
| IFNγ | 100 ng/mL | 472% | 4631% |
| Retinoic acid | 0.005% | 26% | 161% |
| Retinol | 0.01% | 168% | 17 562% |

It is observed that retinoic acid at 0.005% weakly stimulates hBD3 synthesis and that retinol (or vitamin A) weakly stimulates hBD2 and strongly stimulates hBD3.

In order to establish whether these products induce irritation or intolerance reactions, three cosmetic formulations were made up according to example 4, using the following variations:
Placebo cream A: no product was added to the formulation: the “Products of the Invention” according to this exemples are not added to the formulation
Cream B: the “Product of the Invention” according to this example is retinoic acid and the concentration used in the formula is 0.005%.
Cream C: the “Product of the Invention” according to this example is retinol and the concentration used in the formula is 0.01%.
   These three formulations were tested in two different ways:
1) By repeated application to animals in order to determine the primary skin irritation index of the preparations.
2) By repeated patch application to human volunteers in order to determine the irritant or sensitizing potential of the formulations.

With these three formulations as used in both studies, no irritation nor allergy was found and retinoic acid and retinol (as well their precursors and derivatives) at the concentrations used can therefore be regarded as not inducing any inflammatory, irritation or intolerance reaction.

### EXAMPLE 4

### Anti-Wrinkle Cream

| INCI Name | Quantity |
|---|---|
| WATER | add 100 |
| GLYCERIN | 5 |
| CARBOMER ® | 0.2 |
| TETRASODIUM EDTA | 0.1 |
| CAMELLIA SINENSIS LEAF OIL | 2 |
| HYDROGENATED POLYISOBUTENE | 8 |
| GLYCERYL STEARATE SE | 2 |
| DIMETHICONE | 1 |
| GLYCERYL STEARATE AND PEG-100 STEARATE | 1.5 |
| TRIETHYLHEXANOINE | 5 |
| STEARIC ACID | 2 |
| CETYL ALCOHOL | 1 |
| SILICA | 1 |
| DICAPRYLYL MALEATE | 6 |
| GLYCERYL STEARATE | 1 |
| DIMETHICOME | 3.5 |
| WATER | 2.3 |
| TRIETHANOLAMINE | 0.5 |
| PHENOXYETHANOL, METHYLPARABEN, | 0.7 |
| PROPYLPARABEN, BUTYLPARABEN, | |
| ETHYLPARABEN | |
| SCENT | 0.3 |
| TOCOPHEROL ACETATE | 0.5 |
| RETINOL | 0.1 |
| SODIUM HYALURONATE | 0.03 |
| CENTELLA ASIATICA EXTRACT | 1 |
| HYDROLYSED SOYA PROTEIN | 0.4 |
| PRODUCTS OF THE INVENTION | 0.001 to 20 |

### EXAMPLE 5

### Anti-Spot Serum

| INCI Name | Quantity |
|---|---|
| WATER | add 100 |
| TRISODIUM EDTA | 0.1 |
| HYDROXYMETHYLCELLULOSE | 0.1 |
| XANTHAN GUM | 0.3 |
| PHENOXYETHANOL, METHYLPARABEN, | 0.56 |
| PROPYLPARABEN, BUTYLPARABEN, | |
| ETHYLPARABEN | |
| BUTYLENE GLYCOL | 5 |
| POLYSORBATE 20 | 1 |
| SCENT | 0.05 |
| TOCOPHEROL ACETATE | 0.1 |
| SODIUM CITRATE | 0.65 |
| MAGNESIUM ASCORBYL PHOSPHATE | 1 |
| PRODUCTS OF THE INVENTION | 0.001 to 20 |

### EXAMPLE 6

### Foundation

| INCI Name | Quantity |
|---|---|
| WATER | add 100 |
| MAGENSIUM ALUMINIUM SILICATE | 0.5 |
| CELLULOSE GUM | 0.35 |
| GLYCERIN | 3 |
| POLYVINYL PYRROLIDONE | 5 |
| DIPROPYLENE GLLYCOL | 0.05 |
| PROPYLENE GLYCOL | 2 |
| PHENOXYETHANOL, METHYLPARABEN, | 1 |
| PROPYLPARABEN, BUTYLPARABEN, | |
| ETHYLPARABEN | |
| XANTHAN GUM | 0.2 |
| TRIETHANOLAMINE | 0.7 |
| ISOPROPYL PALMITATE | 4 |
| MINERAL OIL | 2 |
| HEXYLDECANOL | 3 |
| GLYCERYL STEARATE | 2 |
| STEARIC ACID | 2.4 |
| OLEIC ACID | 0.5 |
| POLYSORBATE 80 | 0.7 |
| TOCOPHEROL | 0.5 |
| TITANIUM DIOXIDE | 7 |
| IRON OXIDE | 4 |
| NYLON-2 | 6 |
| SODIUM HYALURONATE | 0.01 |
| SCENT | 0.05 |
| PROPYLENE GLYCOL DICAPRYLATE/DICAPRATE | 7.2 |
| PROUCTS OF THE INVENTION | 0.001 to 20 |

### EXAMPLE 7

### UVA/UVB Protective Whitening Cream

| INCI Name | Quantity |
|---|---|
| OCTYLMETHOXYCINNAMATE | 4 |
| CETHYL PEG/PPG-10/1DIMETHICONE | 3 |
| Bis-PEG/PPG-14/14 DIMETHICONE | 3 |
| DIMETHICONE | 6 |
| CYCLOMETHICONE | 4 |
| POLYDECENE | 4 |
| PHENOXYETHANOL, METHYLPARABEN, | 0.65 |
| PROPYLPARABEN, BUTYLPARABEN, | |
| ETHYLPARABEN | |
| TOCOPHEROL | 0.5 |
| SCENT | 0.8 |
| PPG-3 MYRISTYL ETHER | 0.5 |
| TITANIUM DIOXIDE | 8 |
| PHENOXYETHANOL | 0.35 |
| SODIUM CITRATE | 0.65 |
| MAGNESIUM ASCORBYL PHOSPHATE | 3 |
| WATER | QSP 100 |
| XANTHAN GUM | 0.4 |
| BUTYLENE GLYCOL | 2 |
| PRODUCTS OF THE INVENTION | 0.001 to 20 |

### EXAMPLE 8

### Slimming Gel

| INCI Name | Quantity |
|---|---|
| WATER | add 100 |
| TETRASODIUM EDTA | 0.2 |
| CARBOMER ® | 0.5 |
| GLYCERIN | 3.0 |
| POLYGLYCERYLMETHACRYLATE AND | 5.0 |
| PROPYLENE GLYCOL | |
| DIPROPYLENE GLYCOL | 3.0 |
| BUTYLENE GLYCOL | 5.0 |
| PHENOXYETHANOL, METHYLPARABEN, | 0.65 |
| PROPYLPARABEN, BUTYLPARABEN, | |
| ETHYLPARABEN | |
| TRIETHANOLAMINE | 0.5 |
| CAFFEINE | 2 |
| RUSCUS ACULEATUS EXTRACT | 1 |
| PRODUCTS OF THE INVENTION | 0.001 to 20 |

### EXAMPLE 9

### Moisturizer Cream

| INCI Name | Quantity |
|---|---|
| WATER | add 100 g |
| CARBOMER ® | 0.35 |
| TETRASODIUM EDTA | 0.1 |
| POLYSORBATE 60 | 3 |
| SORBITAN STEARATE | 2.6 |
| ISOPROPYL PALMITATE | 2.5 |
| CETYL PALMITATE | 4 |
| ETHYLHEXYL PALMITATE | 5.1 |
| SQUALENE | 1 |
| CETHYL ALCOHOL | 2.5 |
| CYCLOMETHICONE | 1 |
| DIMETHICONE | 0.5 |
| TRIETHANOLAMINE | 0.53 |
| BUTYLENE GLYCOL | 5 |
| SCENT | 0.3 |
| PHENOXYETHANOL, METHYLPARABEN, | 0.65 |
| PROPYLPARABEN, BUTYLPARABEN, | |
| ETHYLPARABEN | |
| TOCOPHERAL ACETATE | 0.5 |
| SODIUM HYALURONATE | 0.03 |
| SWEET ALMOND OIL PROTEIN AND GLYCERIN | 2 |
| POLYGLYCERYLMETHACRYLATE | 5 |
| PRODUCTS OF THE INVENTION | 0.001 to 20 |

### EXAMPLE 10

### Shampoo

| INCI Name | Quantity |
|---|---|
| WATER | add 100 g |
| XANTHAN GUM | 0.8 |
| CITRIC ACID | 0.8 |
| SODIUM LAURETH SULFATE | 40 |
| PHENOXYETHANOL, METHYLPARABEN, | 2 |
| PROPYLPARABEN, BUTYLPARABEN, | |
| ETHYLPARABEN | |
| PRODUCTS OF THE INVENTION | 0.001 to 20 |

1. (canceled)

2. A method of exerting a bactericidal and/or fungicidal effect on a tissue, said method comprising topically applying on a tissue of a subject in need thereof a cosmetic composition comprising between 0.1% and 1% (w/w) of an aqueous boldo extract obtained by extraction in water and at least one cosmetically acceptable excipient, wherein said cosmetic composition is applied in an amount effective to exert a bactericidal and/or fungicidal effect on the tissue and to stimulate expression of human beta-defensin 2 (hBD2) and/or human beta-defensin 3 (hBD3) in the tissue.

3. The method of claim 2, wherein the amount of the cosmetic composition applied does not act to stimulate inflammatory, irritation, or intolerance reactions in the tissue.

4. The method of claim 2, wherein the tissue is skin, hair, nails, and/or scalp.

5. The method of claim 2, wherein the tissue is inflicted by a disorder selected from the group consisting of dandruff, acne, vitiligo, bacterial dermatosis, and fungicidal dermatosis.

6. The method of claim 2, wherein said cosmetic composition further comprises at least one microbiocidal and/or microbio static agent.

7. The method of claim 2, wherein said cosmetic composition comprises 0.1% (w/w) of the aqueous boldo extract.

8. The method of claim 2, wherein said cosmetic composition comprises 0.5% (w/w) of the aqueous boldo extract.

9. The method of claim 2, wherein said cosmetic composition comprises 1% (w/w) of the aqueous boldo extract.

10. The method of claim 2, wherein the cosmetic composition is adapted to a galenic form selected from the group consisting of an aqueous solution, a lotion-type dispersion, a two-phase dispersion, a water/oil or oil/water emulsion, a triple emulsion, a vesicle dispersion, creams, milks, liquid soaps, a body emulsion, dermatological bars, a serum, a mousse, an aerosol, a stick, a make-up product for the skin, and a cleansing product for the skin, hair, nails, and/or scalp.


SEQUENCE LISTING (6 sequences)

SEQ ID NO: 1 - DNA, 20 bp, Artificial sequence
  Primer
gtggggcgccccaggcacca

SEQ ID NO: 2 - DNA, 24 bp, Artificial sequence
  Primer
ctccttaatgtcacgcacgatttc

SEQ ID NO: 3 - DNA, 21 bp, Artificial sequence
  Primer
ccagccatcagccatgagggt

SEQ ID NO: 4 - DNA, 21 bp, Artificial sequence
  Primer
ggagccctttctgaatccgca

SEQ ID NO: 5 - DNA, 21 bp, Artificial sequence
  Primer
agcctagcagctatgaggatc

SEQ ID NO: 6 - DNA, 21 bp, Artificial sequence
  Primer
cttcggcagcattttcggcca